(19) United States
(12) Patent Application Publication
(10) Pub. No.: US 2012/0071431 A1
(43) Pub. Date: Mar. 22, 2012

(54) Platycodin Radix Extracts, and Their Use in Treating Disease Conditions Associated with Angiogenesis

(76) Inventors: Frank L. Greenway, Baton Rouge, LA (US); Zhijun Liu, Baton Rouge, LA (US)

(21) Appl. No.: 13/073,135

(22) Filed: Mar. 28, 2011

Related U.S. Application Data
(60) Provisional application No. 61/318,435, filed on Mar. 29, 2010.

(51) Int. Cl. A61K 31/704; A61P 7/06; A61P 35/00; A61P 31/04; A61P 19/04; A61P 25/00; A61P 19/08; A61P 7/00; A61P 17/06; A61P 29/00; A61P 27/10; A61P 33/02; A61P 27/02; A61P 1/00

(52) U.S. Cl. 514/33

(57) ABSTRACT

Extracts of radix platycodin are used to inhibit angiogenesis, particularly angiogenesis that is associated with disease conditions other than obesity or cancer.

The benefit of the Mar. 29, 2010 filing date of provisional patent application Ser. No. 61/318,435 is claimed under 35 U.S.C. §119(e).

This invention pertains to platycodin radix extracts, and to their use in treating disease conditions associated with angiogenesis.

Angiogenesis

In an adult, two types of blood vessels can potentially be found. The normal blood vessel is a resting, quiescent, fully developed vessel. A second form, a proliferating or developing blood vessel, occurs rarely during the normal life cycle (only in early development and reproduction, e.g., menstrual cycle and pregnancy). In contrast, the process of angiogenesis, the proliferation and development of new blood vessels, often occurs in wound healing and in pathological processes, e.g., tumor growth. Angiogenesis is a complex process involving many stages, including extracellular matrix remodeling, endothelial cell migration and proliferation, capillary differentiation, and anastomosis. All detectable solid tumors (tumors over 2 mm in diameter) exploit angiogenesis to supply the needed blood to proliferating tumor cells. Studies have demonstrated that the level of vascularization in a tumor is strongly associated with metastasis in melanoma, breast, and lung carcinomas. See R. Bicknell, “Vascular targeting and the inhibition of angiogenesis,” Annals of Oncology, vol. 5, pp. 45-50 (1994).

Angiogenesis inhibitors have been suggested to intervene into neoplastic processes. See G. Gasparini, “The rationale and future potential of angiogenesis inhibitors in neoplasia,” Drugs, vol. 58, pp. 17-38 (1999). The inhibitory agents block angiogenesis, thereby causing tumor regression in various types of neoplasia. Known therapeutic candidates include naturally occurring angiogenesis inhibitors (e.g., angiostatin, endostatin, platelet factor-4), specific inhibitors of endothelial cell growth (e.g., TNP-470, thalidomide, interleukin-12), agents that neutralize angiogenic molecules (e.g., antibodies to fibroblast growth factor or vascular endothelial growth factor), suramin and its analogs, tecogalan, agents that neutralize receptors for angiogenic factors, agents that interfere with vascular basement membrane and extracellular matrix (e.g., metalloprotease inhibitors, angiostatic steroids), and anti-adhesion molecules (e.g., antibodies such as anti-integrin alpha V beta 3). See L. Rosen, “Antiangiogenic strategies and agents in clinical trials,” Oncologist, vol. 5, supplement 1, pp. 20-27 (2000).

Abnormal angiogenesis occurs when improper control of angiogenesis causes either excessive or insufficient blood vessel growth. Excessive blood vessel proliferation promotes outcomes such as tumor growth, development of distant metastases, blindness, skin disorders such as psoriasis, and rheumatoid arthritis. Diseases that have been associated with neovascularization include, for example, Crohn's disease, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, mycobacterial infections, Lyme disease, systemic lupus erythematosis, psoriasis, retinopathy of prematurity, Eales disease, Bechets disease, infections causing retinitis or choroiditis, presumed ocular histoplasmosis, Bests disease, myopia, optic pits, Stargarts disease, pars planitis, chronic retinal detachment, hyperviscosity syndrome, toxoplasmosis, trauma, and post-laser complications. Other angiogenic-related diseases may include, for example, diseases associated with rubeosis (neovascularization of the angle), and diseases caused by abnormal proliferation of fibrovascular or fibrous tissue, including all forms of proliferative vitreoretinopathy. Any disease having a known angiogenic counterpart could potentially be treated with an anti-angiogenic factor, e.g., psoriasis. See D. Creamer et al., “Overexpression of the angiogenic factor platelet-derived endothelial cell growth factor/thymidine phosphorylase in psoriatic epidermis,” Br. J. Dermatol., vol. 137, pp. 851-855 (1997).

Angiogenesis is a prominent contributor to solid tumor growth and the formation of distant metastases. Several experimental studies have concluded that primary tumor growth, tumor invasiveness, and metastasis all require neovascularization. The process of tumor growth and metastasis is complex, involving interactions among transformed neoplastic cells, resident tissue cells (e.g., fibroblasts, macrophages, and endothelial cells), and recruited circulating cells (e.g., platelets, neutrophils, monocytes, and lymphocytes). A possible mechanism for the maintenance of tumor growth is an imbalance, or disregulation, of stimulatory and inhibitory growth factors in and around the tumor. Disregulation of multiple systems allows the perpetuation of tumor growth and eventual metastasis. Angiogenesis is one of many systems that is disregulated in tumor growth. In the past it has been difficult to distinguish between disregulation of angiogenesis and disregulation of other systems affecting a developing tumor. Another complicating factor is that aggressive human melanomas mimic vasculogenesis by producing channels of patterned networks of interconnected loops of extracellular matrix, in which red blood cells, but not endothelial cells, are detected. See A. J. Maniotis et al., “Vascular channel formation by human melanoma cells in vivo and in vitro: Vasculogenic mimicry,” Am. J. Pathol., vol. 155, pp. 739-52 (1999). These channels may facilitate perfusion of tumors, independent of perfusion by angiogenesis.

A tumor cannot expand beyond approximately 2 mm without a blood supply to provide nutrients and remove cellular wastes. Solid tumors in which angiogenesis is important include malignant tumors, and benign tumors including acoustic neuroma, neurofibroma, trachoma, and pyogenic granulomas. Inhibiting angiogenesis could halt growth and potentially lead to regression of these tumors. Angiogenic factors have been reported as being associated with several solid tumors, including rhabdomyosarcoma, retinoblastoma, Ewing sarcoma, neuroblastoma, and osteosarcoma.

Angiogenesis has also been associated with some non-solid tumors, including blood-born tumors such as leukemias, which are various acute or chronic neoplastic diseases of the bone marrow marked by unrestrained proliferation of white blood cells, usually accompanied by anemia, impaired blood clotting, and enlargement of the lymph nodes, liver, and spleen. It is believed that angiogenesis may play a role in the abnormalities in the bone marrow that give rise to leukemias and multiple myelomas.

Antiangiogenic factors can inhibit tumor growth beyond 2 mm by inhibiting the angiogenic response and thus inhibiting blood vessel growth to the tumor. Although angiogenesis in a tumor may begin at an early stage, a tumor requires a blood supply to grow much beyond about 2 mm. Up to 2 mm diameter, tumors can survive by obtaining nutrients and oxygen by simple diffusion. Most anti-angiogenic factors are not cytotoxic, i.e., capable of killing the tumor cells directly. Small tumors of a size about 1 mm³ can be effectively inhibited and destroyed by factors, either endogenous or exogenous, that stimulate the immune system. It is generally accepted that once a tumor has reached a critical size, the immune system is no longer able to effectively destroy the tumor; i.e., there is a negative correlation between tumor size and immune competence. See A. K. Eerola et al., “Tumour infiltrating lymphocytes in relation to tumour angiogenesis, apoptosis,” Lung Cancer, vol. 26, pp. 73-83 (1999); and F. A. Wenger et al., “Tumor size and lymph-node status in pancreatic carcinoma—is there a correlation to the preoperative immune function?” Langenbecks Archives of Surgery, vol. 384, pp. 473-478 (1999). Early adjuvant use of an effective anti-angiogenic agent to preclude development of tumor metastases beyond 1 to 2 mm³ may allow more effective tumor attack and control by the body's immunological mechanisms. In addition, prolonged adjuvant use of a non-toxic angiogenic inhibitor may prevent tumor dissemination by blocking the growth of vessels required for the transport of tumor cells that would form metastatic foci.

Platycodin Radix

Dietary herbal supplements are foods, and as such are generally recognized as safe. Platycodi radix is a name for the roots of Platycodin grandiflorum (Jacq.) A.DC., a plant that is also known by the common name of “balloon flower”. It is used in recipes of Kimchi, for example. It is used as an herbal medicine to treat colds, and there is a belief in Korea that it can prevent obesity It is a form of radish commonly sold in Korean markets. It grows in northern Asia (China, Japan and Russia). This vegetable has been used in traditional oriental medicine to treat colds and respiratory ailments. It is also thought to have anti-diabetic properties by enhancing insulin sensitivity in the liver and improving glucose stimulated insulin secretion. It has anti-inflammatory properties thought to be due to its inhibition of nuclear factor-kappa B. It has anti-cancer properties as well, due to stimulation of apoptosis in colon cancer cells. The platycodin saponins are a constituent of this vegetable. The platycodin saponins, particularly platycodin D, have been shown to inhibit pancreatic lipase, reduce liver fat, reduce serum triglycerides and cause weight loss in rodents. The pickled roots of Platycodin grandiflorum have been used in China and Korea to prevent obesity, but clinical trials have not been reported.

Zhao et al., Antiobese and hypolipidemic effects of platycodin saponins in diet-induced obese rats: evidence for lipase inhibition and calorie restriction. Int J Obeso (Lond.) 2005; 29(8):983-90 performed a study in rats that was particularly instructive. Platycodin saponins or saline were gavage-fed to Sprague-Dawley rats for 4 weeks on a high fat diet after 4 weeks of pretreatment with the same diet. There were 4 groups—one receiving a high fat diet containing 25% beef tallow, one receiving a normal fat diet without beef tallow, one receiving the high fat diet with 35 mg/kg body weight of platycodin saponins per day, and one receiving the high fat diet with 70 mg/kg body weight per day of platycodin saponins. The two platycodin saponin groups had 13±4% less weight gain than either of the control diet groups. The rise in fecal triglyceride was dose dependent, and increased in the 35 and 70 mg per kg per day groups 2.1 to 3.2 times control, respectively. The serum triglycerides and cholesterol fell 28-24% and 41-52%, respectively in the 30 and 70 mg per kg per day groups compared to the high-fat control. Food intake was dramatically reduced. In the first week food intake decreased 42-67%. The food intake was correlated to the decrease in body weight (r=0.75, p<0.005). The lipase inhibition of the individual saponins in the fraction was identical, suggesting that the entire saponin fraction was responsible for the lipase inhibition. There were no adverse events or changes in the feces.

We have discovered that extracts of radix platycodin can be used to inhibit angiogenesis, particularly angiogenesis that is associated with conditions other than obesity or cancer.

We tested the extract of radix platycodin in our human fat angiogenesis assay and found that it strongly inhibited angiogenesis. General Nutrition Corporation (GNC) provided an extract of platycodin radix standardized to platycodin D. We wanted to show that the active ingredients are absorbed and the serum is effective in inhibiting angiogenesis in our human fat angiogenesis assay. See Greenway F L, Liu Z, Yu Y, Caruso M K, Roberts A T, Lyons J, Schwimer J E, Gupta A K, Bellanger D E, Guillot T S, Woltering E A. An assay to measure angiogenesis in human fat tissue. Obes Surg. 2007 April; 17(4):510-5.

The usual dose of radix platycodin in the treatment of colds is 2-9 grams daily, divided into 3 doses, which has no known health hazards. The only side effect that has been reported is a tranquilizing effect. Radix platycodin is, therefore, usually not taken with excessive alcohol or CNS depressant medications. This study tested a 414 mg dose of platycodin radix saponins. This dose was derived from the dose given to rats in the study by Zhao et al. (2005) for lipase inhibition and calorie restriction, converted to a human dose using a metabolic mass equation.

### BRIEF DESCRIPTION OF THE DRAWINGS

FIG. 1 depicts a chromatographic fingerprint of platycodin root extract containing platycodin saponin (PS). Platycodin D (PD), one of the PS's, eluted at 28 minutes. The PD concentration in the PS was 0.2% w/w.

FIG. 2 depicts an enlarged chromatographic fingerprint from 20 min. to 36 min., of platycodin saponins (PS) containing platycodin D (PD) and other saponins including platycodin D3 (PD3), polygalactin D (PG), platycodin A (PA), acetyl-PG, prosapogenin D (PRS-D), prosapogenin D methyl ester (PRS-DME), identified by the method of Zhao et al. (2005).

FIG. 3 depicts the molecular structure of platycodin D (PD), a triterpene glucoside.

FIG. 4 depicts the testing of PS in the human fat tissue angiogenesis assay. PS at 1 mg/ml (solid circles) almost completely inhibited angiogenesis as compared to control (open circle).

FIG. 5 depicts a dose-response curve for PD in the human fat tissue angiogenesis assay. At concentrations above 10⁻⁴ M, PD totally inhibited angiogenesis. Concentrations below 10⁻⁴ M did not inhibit angiogenesis.

FIG. 6 depicts the angiogenic index of different concentrations of PD in the human fat tissue angiogenesis assay. Both the 20% and the 40% standardized extracts showed total inhibition of angiogenesis in this assay at concentrations of 10⁻⁴ M and higher.

FIG. 7 depicts the effect on angiogenesis of serum from volunteers who had taken Platycodi radix extract, following various lengths of time. The serum was drawn at time points after 3 fasting volunteers orally took Platycodi radix extract standardized to 414 mg of platycodin D, and was tested in the human adipose tissue angiogenesis assay. The peak reduction in angiogenesis was 26% at 60 minutes (p<0.002). Angiogenesis was reduced by an average of 17.8% between 30 and 240 minutes (p<0.05 to <0.002).

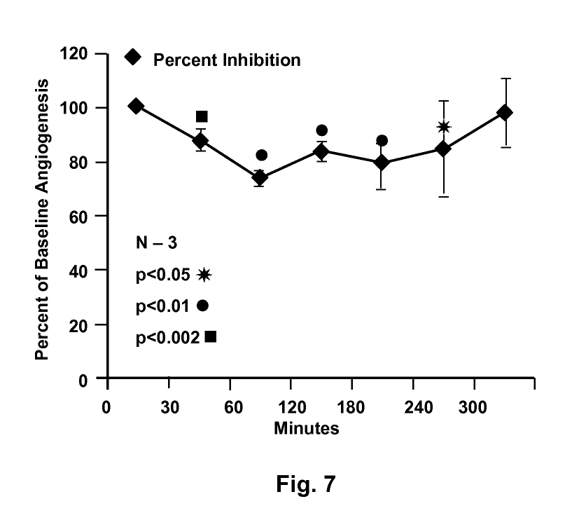

FIG. 8 depicts the inhibition by serum of angiogenesis by oral platycodin radix fasting, versus placebo. The placebo reduced angiogenesis by an average of 5.6%, compared to 17.8% by the platycodi radix extract standardized to platycodin D, between 30 minutes and 240 minutes.

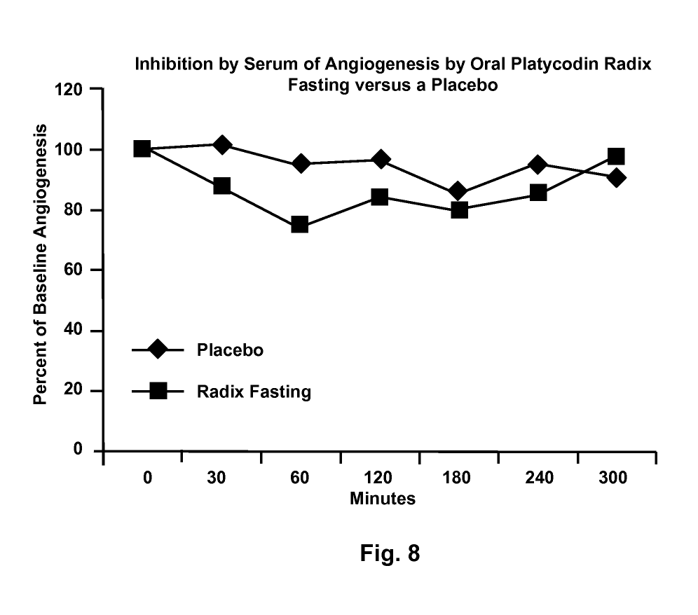

FIG. 9 depicts the inhibition by serum of angiogenesis by oral platycodin radix fasting, versus a standardized meal. Taking the Platycodi radix standardized to platycodin D orally with a 400 kcal meal appeared to delay the absorption of the extract by approximately 3.5 hours.

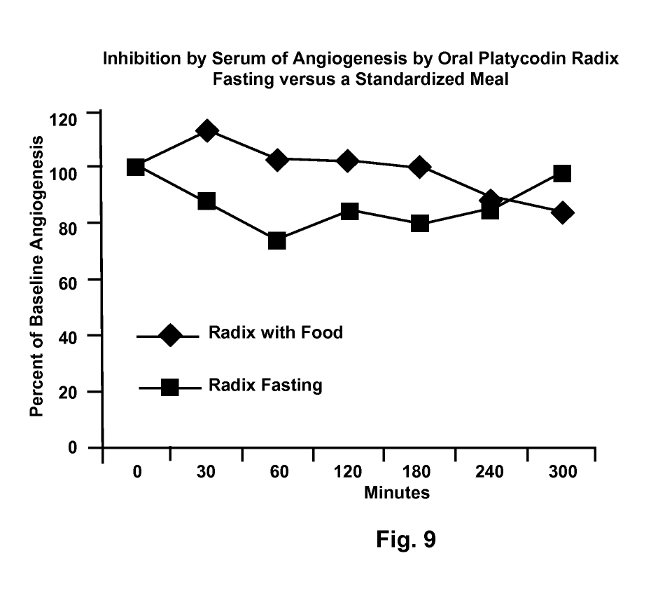

### EXAMPLE 1

Extraction of Platycodin D from Platycodi Radix

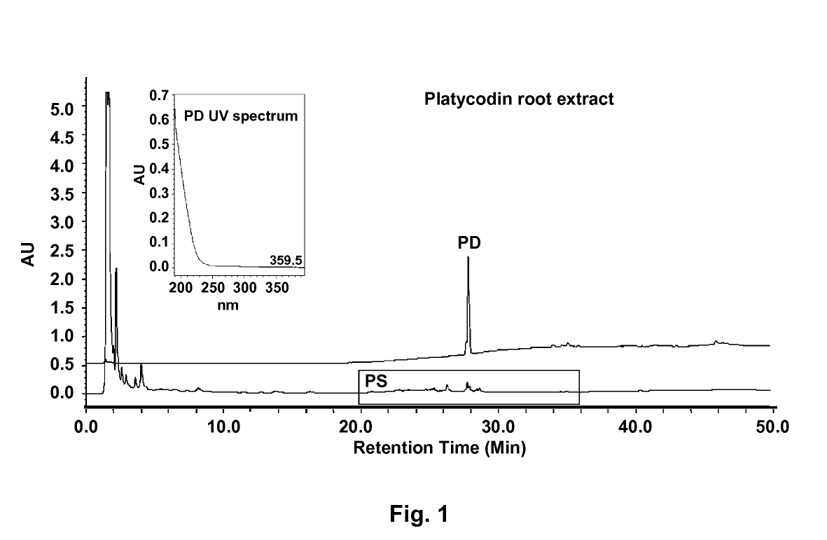

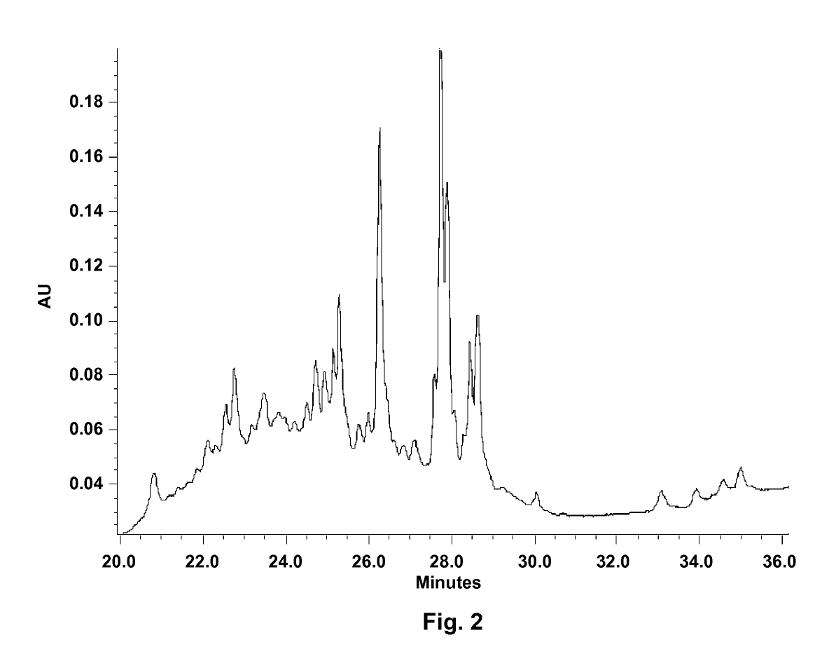

1.75 kg ground powder of Platycodi radix from GNC was rinsed with 8 liters water overnight and extracted with boiling water three times. The aqueous extract (20 liters; FIG. 1) was filtered with a #4 Whatman filter paper before loading on one column containing 10 kg L-493 adsorbent resin to perform SPE chromatography. After 60 liters water was used to elute the column to remove large molecules, presumably mostly polysaccharides and proteins, 20 liters 95% EtOH was used to elute. We collected the EtOH eluate and removed solvent under vacuum before freeze-drying to yield 190 g platycodi saponins (PS; FIG. 2) (10.8% w/w yield).

Isolation of Platycodin D (PD)

Step 1. Silica gel: 140 g PS was dissolved into 50% MeOH and mixed with 140 g silica gel (200-300 mesh) and dried. The samples were chromatographed with 1.6 kg silica gel. Mobile phase (CHCl₃ and MeOH in gradient, from 8:1 to 2:1) was used as eluent to get 9 fractions. The fractions were examined with TLC and HPLC. Fractions 4-5 contained PD.

Step 2. Flash chromatography: Above fraction was subjected to silica gel flash chromatography. Mobile phase (CHCl₃ and MeOH in gradient, from 8:1 to 2:1) was used to yield 9 fractions. The fractions were examined with TLC and HPLC. Fractions 4-7 contained PD.

Step 3. Sephadex LH-2: Fractions 4˜7 were separated on Sephadex LH-20 to yield fractions. The fractions were checked with TLC method. The fractions containing PD were combined.

Step 4. Preparative TLC: The fractions were separated by preparative TLC. TLC plates (20×20 cm, 1.5 mm thick, Ana) and n-BuOH-Acetic acid-H₂O=4:1:1 were used. Then we collected the gel of the area of Rf=0.4, and washed with 400 ml methanol.

Step 5. Preparative HPLC: Above combined fraction was run on prep-HPLC to collect the fraction between 8 and 15 min.

NMR and MS Analysis

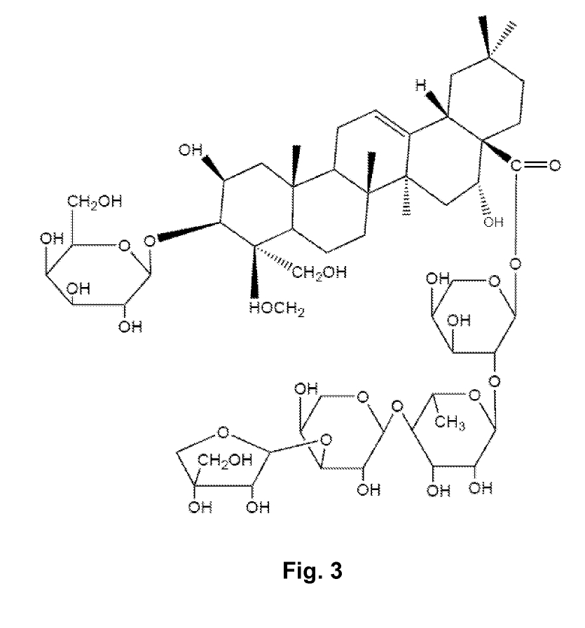

20 mg compound final fraction was dissolved into 0.5 ml pyridine-d5 for NMR analysis. 1 mg compound was analyzed by MALDI MS. MALDI-TOF spectrum showed peaks at m/z 1247 (M+Na) and 1263 (M+K) to indicate PD's MW is 1224. ¹³C-NMRand ¹H-NMR spectrum showed the exact same spectrum as reference platycodin D. (Reference data: Axihiro Tada, Yoshio Kaneiwa, Junzo Shoji, et al. Studies on the saponins of the roots of Platycodin grandiflorum and isolation and the structure of platycodin D. Chem. Pharm Bull, 1975, 23(11): 2965-2972). Based on the result including TLC, HPLC, MS and NMR data, compound X was elucidated as platycodin D (FIG. 3).

Antiangiogenic Activity of PS and PD Fractions

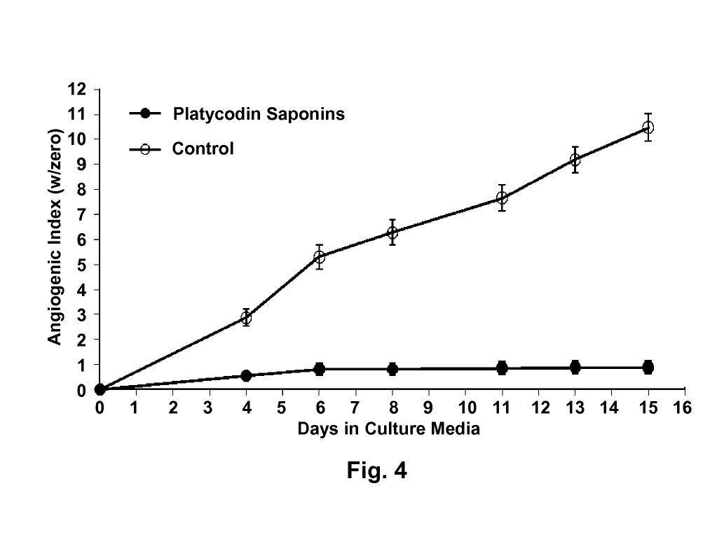

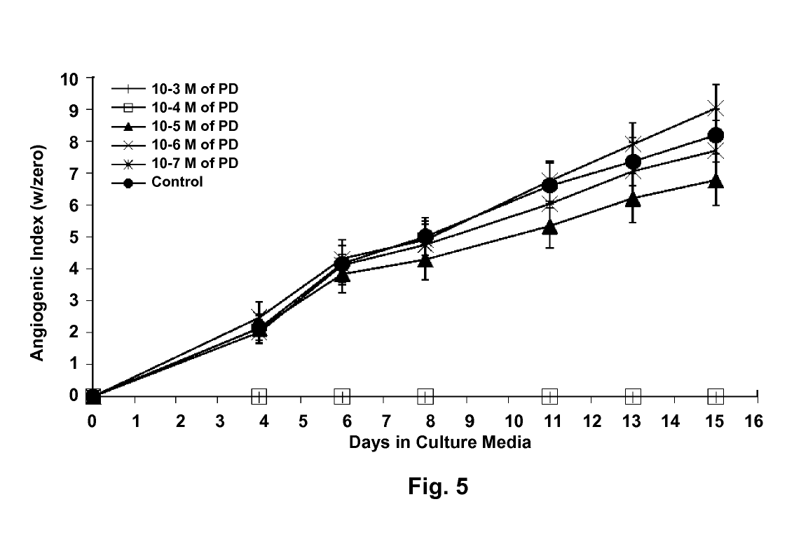

The above extracts of platycodin, both PS and PD, were assayed for antiangiogenic activity using the human fat angiogenesis assay of Greenway et al. (2007). Results are shown in FIGS. 4 and 5.

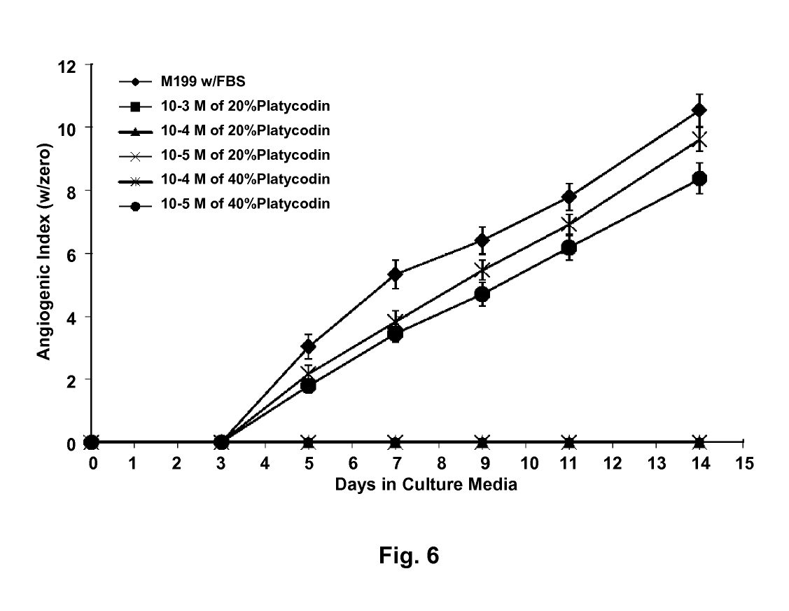

In addition, the platycodin D (PD) isolated from platycodin saponins was sent to General Nutrition Corporation (GNC). GNC used the platycodin D to standardize an extract of platycodin radix, also known as balloon flower, to 20% and 40% platycodin D. These standardized extracts were then tested for anti-angiogenic activity in the same human fat angiogenesis assay. The results are shown in FIG. 6. Both standardized extracts totally inhibited angiogenesis at a concentration of 10⁻⁴ M and higher.

### EXAMPLE 2

Anti-Angiogenic Activity of Plasma Platycodin Radix Extract Administered Orally

Human subjects were given a placebo or 414 mg of platycodin radix saponins orally, with or without food. Blood was drawn at times 0, 30, 60, 120, 180, 240, and 300 minutes. The serum samples were then tested in the human fat angiogenesis assay of Greenway et al. (2007).

The inclusion criteria for the subjects were healthy male or female, 18-65 years of age, inclusive; and BMI less than 35 kg/m². Subjects were excluded if pregnant or nursing, if taking any chronic medication other than oral contraceptives or hormone replacement; or if of childbearing potential and unwilling to avoid pregnancy during the study.

Study Design: The study consisted of 2 screening visits and 1 study visit. The study visit was followed by a short clinic visit for safety testing on the following morning. The first screening visit consisted of a chemistry panel and CBC. The second screening visit consisted of a medical history, physical examination and electrocardiogram. Five subjects participated in this study, and each had 1 test day starting at 8 am.

All subjects reported on the test day having nothing to eat or drink except water from 9 pm the prior night. Three subjects were given, without food, a 414 mg dose of platycodin D either using the 20% or 40% extract of platycodin radix standardized by GNC to platycodin D. One subject was given placebo without food. One subject was given the 414 mg dose on one occasion with a standard breakfast, consisting of water and a 400 kcal omelet made of egg, butter, flour, and dried onions with a macronutrient composition of 40% fat, 40% carbohydrate, and 20% protein.

On the test days, subjects had an intravenous line placed, and 50 milliliters of blood were withdrawn at 0 minutes. Subjects then consumed the extract of Platycodin radix in capsule form with or without the standard breakfast. The assignment to treatment condition was done randomly and the study was double blinded. Fifty milliliters of blood was drawn from the intravenous line at 30, 60, 120, 180, 240 and 300 minutes. The serum was separated from the blood samples and frozen at −70° C. until analyzed in the angiogenesis assay. Subjects returned on the morning after the test day having nothing to eat or drink except water from 9 pm the prior night for a CBC, chemistry panel and an electrocardiogram, with questioning about any adverse events.

This was a pharmacokinetic study to evaluate the absorption of the extract of platycodin radix, retention of its anti-angiogenic activity, timing of its peak serum level and its biological half-life in serum.

The standardized extract administered orally was shown to be effective in inhibiting angiogenesis in vivo. Serum from the three people given a standardized Platycodin radix extract orally inhibited angiogenesis in human fat tissue by a maximum of about 25% from about 30 min to 240 min. When the extract was taken with food, the meal delayed the absorption of platycodin D and slowed the time until anti-angiogenic activity was seen.

Thus, oral administration of this Platycodin radix extract was shown to cause anti-angiogenic activity in the serum. The extract can be used to treat diseases that are associated with angiogenesis and that were previously unknown to be affected by Platycodin radix or its extracts, including without limitation psoriasis, retinopathy, and rheumatoid arthritis.

### EXAMPLE 3

Pharmacokinetic Study of the Antiangiogenic Activity of Standardized Platycodi Radix

(Example 3 is an alternative description of the same experiments and data discussed above for Example 2. There is partial overlap in these alternative descriptions.)

Methods summary: We tested Platycodi radix extract, platycodin D, and an extract of Platycodi radix standardized to platycodin D for their ability to inhibit angiogenesis in a human adipose tissue assay. We treated 5 healthy volunteers orally with platycodi radix extract standardized to 414 mg of platycodin D under fasting conditions (3 volunteers), with a 400 kcal meal (1 volunteer) or a placebo (1 volunteer), and drew blood over 5 hours to compare serum inhibition of human adipose tissue angiogenesis.

Results summary: Platycodin radix extract, platycodin D, and Platycodi radix extract standardized to platycodin D all inhibited angiogenesis. The 3 volunteers who consumed Platycodi radix extract standardized to 414 mg of platycodin D had a 26% reduction in angiogenesis from baseline at 60 minutes (p<0.002), and had a statistically significant reduction in angiogenesis from 30 to 240 minutes (p<0.05 to p<0.002). The placebo decreased angiogenesis by 5.6% compared to 17.8% by the extract between 30 and 240 minutes. The meal delayed absorption by ˜3.5 hours.

Detailed Methods

Study 1: An aqueous extract of Platycodi radix saponins at 1% w/v was tested in our human adipose tissue assay. Briefly, subcutaneous adipose tissue was removed from patients undergoing cosmetic surgical procedures. The fat was placed directly into sterile assay media, transported directly to the laboratory from the surgery suite in the sterile container and processed under a laminar flow hood. The tissue was cut into fragments approximately 1 mm thick and 2 mm in diameter. These fragments were placed in 96-well plates containing a 4 μL human thrombin solution (0.05 IU in 4 μL per well) and covered with 100 μL clotting media (3 mg/mL fibrinogen; Sigma Chemical Co., St Louis, Mo., USA), 0.5% ε-amino caproic acid (Sigma Chemical Co.) in angiogenesis media containing 100 U/mL penicillin, 100 U/mL streptomycin sulfate and 2.5 μg/mL amphotericin β in Medium 199 (Gibco BRL, Gaithersburg, Md., USA). The mixture was allowed to clot by incubation in 6% CO₂ at 37° C. in a humidified incubator. After the media had gelled overnight, the fat-containing clot was supplemented with 100 μL angiogenesis media containing 20% fetal bovine serum (Gibco BRL). The total volume of each well was 200 μL. There were 30 replicates for the 1% w/v Platycodi radix saponins and the media control. The angiogenesis media were replaced every 48 h and appropriate concentrations of fresh Platycodi radix saponins or media control were added. Wells were evaluated for the angiogenic response as described by Greenway et al. (2007). An observer unbiased to the treatment protocols evaluated the angiogenic response using a semi-quantitative visual rating scale. Briefly, the tissue was viewed under an inverted microscope, visually divided into four quadrants and each quadrant was given a numeric score from 0 to 4 based on the neovessels' length, density, and percentage of the quadrants' circumference involved with the angiogenic response. Numeric results from the four quadrants were summed and expressed as an angiogenic index ranging from 0 (no neovessels apparent in any quadrant) to 16 (highly vascularized in all four quadrants). The extract of Platycodi radix saponins at 1% w/v was tested, followed by Platycodin D, and finally by the Platycodi radix saponins standardized to the Platycodin D content.

Study 2: Five normal healthy volunteers between the ages of 18 and 65 years with a BMI between 18 kg/m² and 35 kg/m² were included in this study. Subjects were excluded if taking regular medication other than oral contraceptives, as were women who were pregnant or nursing, or if of childbearing potential and unwilling to avoid pregnancy during the study. During screening all subjects had a medical history, physical examination, electrocardiogram, a fasting chemistry panel (glucose, creatinine, potassium, uric acid, albumin, calcium, magnesium, creatinine phosphokinase, alanine-leucine transaminase, alkaline phosphatase, iron, cholesterol, triglycerides, high density lipoprotein cholesterol, and low density lipoprotein cholesterol) and a complete blood count (CBC) (hemoglobin, hematocrit, mean cell volume, platelet count, white blood cell count, granulocyte number, neutrophil number, eosinophil number and basophil number). Subjects who passed screening reported for their study visit in the morning after having nothing to eat or drink except for water from 9 pm the prior night. Subjects had an intravenous line placed, from which 50 ml of blood was drawn at times 0, 30, 60, 120, 180, 240 and 300 minutes. After the baseline blood draw 3 subjects took Platycodi radix extract standardized to 414 mg of platycodin D orally, one subject took a placebo orally, and one subject took Platycodi radix extract standardized to 414 mg of platycodin D with a 400 kcal omelet made from egg, butter, flour, and dried onions (40% of energy as fat, 40% as carbohydrate, and 29% as protein). Serum was separated and frozen at −70° C. until analyzed in the angiogenesis assay. Subjects returned on the morning following their test day having had nothing to eat or drink except water from the prior night. Subjects had blood drawn for a chemistry-15 panel, a CBC, an electrocardiogram was performed and the subjects were questioned about any adverse events.

Statistical Analysis

The mean and standard deviation of the percent reduction in angiogenesis from baseline at each time point was compared to baseline by t-test. The time course of the inhibition of angiogenesis was described and the effects of food and placebo on angiogenesis were examined.

Detailed Results

Study 1: Platycodi radix saponins at 1% w/v concentration inhibited angiogenesis (p<0.001). Platycodin D was isolated and tested in the assay over a concentration range from 10⁻⁷ M to 10⁻³ M. Platycodin D gave complete inhibition of angiogenesis at 10⁻⁴ M (p<0.001) and appeared to give partial inhibition at 10⁻⁵M but at 10⁻⁵M the difference was not statistically significant. The platycodi radix extract standardized to platycodin D gave complete inhibition at a 10⁻⁴ M concentration of platycodin D (p<0.001).

Study 2: The 5 normal volunteers had an average BMI of 23.1 kg/m² and an average weight of 63.1 kg. The three subjects who consumed oral platycodi radix extract standardized to 414 mg of platycodin D had a peak reduction of 25.76%±4.93% (p<0.002) in angiogenesis from baseline at 60 minutes. Between 30 minutes and 240 minutes there was a significant reduction of angiogenesis from baseline that varied from 12% to 25.7% (p<0.05 to 0.002) and averaged 17.8%. The average reduction of angiogenesis in the placebo condition between 30 and 240 minutes was 5.6% compared to 17.8% in the platycodi radix extract fasting condition. Since there was only one placebo-treated subject, meaningful statistics could not be done for this comparison. There was only one subject who took the platycodi radix with food. That subject seemed to have a response delayed by approximately 3.5 hours, and at 5 hours when the test ended, the reduction in angiogenesis was still increasing. One subject had moderate nausea and some heartburn after taking the Platycodi radix extract, and another complained of a mild toothache. The nausea and heartburn could have been related to the extract, but the toothache most likely was unrelated. There were no other adverse events during the study. All 5 subjects completed the trial. The lab work on the day following the test day was normal, as was the case at screening, except for the anticipated drop in hemoglobin and hematocrit related to the serial blood draws performed during the test.

Discussion

This pilot study demonstrated that platycodi radix and the platycodin D which it contains are inhibitors of angiogenesis. There was a statistically significant reduction in angiogenesis from baseline after a 414 mg oral dose of platycodi radix from 30 minutes to 240 minutes. Thus, it appears that the platycodi radix extract standardized to its platycodin D content could be dosed three times a day. The standardized platycodi radix extract gave more inhibition of angiogenesis than did placebo, and food seemed to delay absorption by approximately 3.5 hours. There were no serious adverse events, which is not unexpected since platycodi radix itself is a food. Angiogenesis is only typically seen in adulthood for the menstrual cycle, fetal development, and wound healing. Thus, the potential for toxicity is low. The fact that platycodi radix is used as a food is further evidence of its safety.

Miscellaneous

The term “effective amount” as used herein refers to an amount of an extract of Platycodin radix or a component (such as PD) for which, as administered, the resulting plasma concentration is sufficient to inhibit angiogenesis to a statistically significant degree (p<0.05) or to a clinically meaningful degree. The term “effective amount” therefore includes, for example, an amount sufficient to prevent the growth of angiogenic vessels found in diseases of tumor growth, diabetic retinopathy, psoriasis, retinopathy of prematurity, or other angiogenesis-related disease conditions; and preferably to reduce by at least 50%, and more preferably to reduce by at least 90%, the amount of angiogenesis. The dosage ranges for the administration of the extract of Platycodin radix are those that produce the desired effect. Generally, the dosage will vary with the age, weight, condition, sex of the patient or pathology, and the degree of angiogenic response. A person of ordinary skill in the art, given the teachings of the present specification, may readily determine suitable dosage ranges. The dosage can be adjusted in individual cases in the event of any contraindications. In any event, the effectiveness of treatment can be determined by monitoring the extent of angiogenic inhibition or remission by methods well known to those in the field. Moreover, the extract of Platycodin radix can be applied in pharmaceutically acceptable carriers known in the art. The application can be oral, by injection, or topical.

The present invention provides a method of preventing, treating, or ameliorating a disease that is associated with angiogenesis, such as diabetic retinopathy, psoriasis, and the other illustrative conditions described in the specification, comprising administering to a subject at risk for a disease or displaying symptoms for such disease, an effective amount of extract of Platycodin radix. The term “ameliorate” refers to a decrease or lessening of the symptoms or signs of the disorder being treated. The symptoms or signs that may be ameliorated include those associated with an increase in angiogenesis in the body.

In addition to platycodin D, one or more platycodin saponins may be used in practicing this invention. Platycodin saponins are triterpenoid bidesmosides, comprising a triterpenoidal aglycone moiety linked to two glycosyl side chains. Other platycodin saponins include platycodin D3 (PD3), polygalactin D (PG), platycodin A (PA), acetyl-PG, prosapogenin D (PRS-D), prosapogenin D methyl ester (PRS-DME), 2″-O-acetyl polygalactin D, Platycodin A, prosapogenin (PRS), Prs-DME (prs-D methyl ester). See Zhao et al. (2005).

The complete disclosures of all references cited in this specification are hereby incorporated by reference, as is the complete disclosure of priority application 61/318,435. In the event of an otherwise irreconcilable conflict, however, the present specification shall control.

### REFERENCES

1. Obesity: Preventing and managing the global epidemic. Geneva, Switzerland: World Health Organization, 1997.
2. Ogden C L, Carroll M D, Curtin L R, McDowell M A, Tabak C J, Flegal K M. Prevalence of overweight and obesity in the United States, 1999-2004. JAMA. 2006 Apr. 5; 295(13):1549-55.
3. Flegal K M, Carroll M D, Ogden C L, Johnson C L. Prevalence and trends in obesity among US adults, 1999-2000. Jama 2002; 288:1723-7.
4. Bray G A. Obesity: a time bomb to be defused. Lancet 1998; 352:160-1.
5. Skyler J S, Oddo C. Diabetes trends in the USA. Diabetes Metab Res Rev 2002; 18 Suppl 3:S21-6.
6. Ferdinand K C, Clark L T. The epidemic of diabetes mellitus and the metabolic syndrome in African Americans. Rev Cardiovasc Med 2004; 5 Suppl 3:S28-33.
7. Wolf A M, Colditz G A. Current estimates of the economic cost of obesity in the United States. Obes Res 1998; 6:97-106.
8. Hogan P, Dall T, Nikolov P. Economic costs of diabetes in the US in 2002. Diabetes Care 2003; 26:917-32.
9. Clinical Guidelines on the Identification, Evaluation, and Treatment of Overweight and Obesity in Adults—The Evidence Report. National Institutes of Health [published erratum appears in Obes Res 1998 November; 6(6):464] [see comments]. Obes Res 1998; 6 Suppl 2:51 S-209S.
10. Wadden T A, Butryn M L, Byrne K J. Efficacy of lifestyle modification for long-term weight control. Obes Res 2004; 12 Suppl:151 S-62S.
11. Bray G A, Greenway F L. Current and potential drugs for treatment of obesity. Endocr Rev 1999; 20:805-75.
12. Greenway F L. Surgery for obesity. Endocrinol Metab Clin North Am 1996; 25:1005-27
13. Pharmacopoeia of the People's Republic of China (English ed.). Guangzhou, Guangdong Science and Technology Press, 1992.
14. Kwon D Y, Kim S Y, Hong S M, Park S. Long-term consumption of saponins derived from Platycodin radix (22 years old) enhances hepatic insulin sensitivity and glucose-stimulated insulin secretion in 90% pancreatectomized diabetic rats fed a high-fat diet. British J. Nutr. 2009; 101:358-66.
15. Chung J W, Noh E J, Zhao H L, Sim J S, Ha Y W, Shin E M, Lee E B, Cheong C S, Kim Y S. Anti-ionflammatory activity of prosapogenin methyl ester of platycodin D via nuclear factor-kappa B pathway inhibition. Biot Pharm Bull. 2008; 31(11):2114-20.
16. Kim J Y, Park K W, Moon K D, Lee M K, Choi J, Yee S T, Shim K H, Seo K I. Induction of apoptosis in HT-29 colon cancer cells by crude saponin from platycodin radix. Food Chem. Toxicol. 2008; 46(12):3753-8.
17. Han L K, Xu B J, Kimura Y, Zheng Y, Okuda H. Platycodin radix affects lipid metabolism in mice with high fat diet-induced obesity. J Nutr 2000; 130:2760-4.
18. Han L K, Zheng Y N, Xu B J, Okuda H, Kimura Y. Saponins from platycodin radix ameliorate high fat diet-induced obesity in mice. J Nutr 2002; 132:2241-5.
19. Zhao H L, Sims J S, Shim S H, HA Y W, Kang S S, Kim Y s. Antiobese and hypolipidemic effects of platycodin saponins in diet-induced obese rats: evidence for lipase inhibition and calorie restriction. Int J Oges (lond). 2005; 29(8):983--90.
20. Greenway F L, Liu Z, Yu Y, Caruso M K, Roberts A T, Lyons J, Schwimer J E, Gupta A K, Bellanger D E, Guillot T S, Woltering E A. An assay to measure angiogenesis in human fat tissue. Obes Surg. 2007 April; 17(4):510-5.
21. Platycodin Grandiflorum. Third ed. Mountvale, 2004.
22. Heusner A A. Energy metabolism and body size. I. Is the 0.75 mass exponent of Kleiber's equation a statistical artifact? Respir Physiol 1982; 48:1-12.
23. Low S, Chin M C, Deurenberg-Yap M. Review on epidemic of obesity Ann Acad Med Singapore. 2009 Jan; 38(1):57-9.
24. Flegal K M, Carroll M D, Ogden C L, Curtin L R. Prevalence and trends in obesity among US adults, 1999-2008. JAMA. 2010 Jan. 20; 303(3):235-41.
25. Flegal K M, Carroll M D, Ogden C L, Johnson C L. Prevalence and trends in obesity among US adults, 1999-2000. Jama 2002; 288:1723-7.
26. Bray G A. Obesity: a time bomb to be defused. Lancet 1998; 352:160-1.
27. Skyler J S, Oddo C. Diabetes trends in the USA. Diabetes Metab Res Rev 2002; 18 Suppl 3:S21-6.
28. Ferdinand K C, Clark L T. The epidemic of diabetes mellitus and the metabolic syndrome in African Americans. Rev Cardiovasc Med 2004; 5 Suppl 3:S28-33.
29. Wolf A M, Colditz G A. Current estimates of the economic cost of obesity in the United States. Obes Res 1998; 6:97-106.
30. Hogan P, Dall T, Nikolov P. Economic costs of diabetes in the US in 2002. Diabetes Care 2003; 26:917-32.
31. Clinical Guidelines on the Identification, Evaluation, and Treatment of Overweight and Obesity in Adults—The Evidence Report. National Institutes of Health [published erratum appears in Obes Res 1998 November; 6(6):464] [see comments]. Obes Res 1998; 6 Suppl 2:51 S-209S.
32. Wadden T A, Butryn M L, Byrne K J. Efficacy of lifestyle modification for long-term weight control. Obes Res 2004; 12 Suppl:151 S-62S.
33. Bray G A, Greenway F L. Current and potential drugs for treatment of obesity. Endocr Rev 1999; 20:805-75.
34. Greenway F L. Surgery for obesity. Endocrinol Metab Clin North Am 1996; 25:1005-27
35. Pharmacopoeia of the People's Republic of China (English ed.). Guangzhou, Guangdong Science and Technology Press, 1992.
36. http://www.sumobrain.com/patents/ip/Method-producing-platycodi-radix-root/JP2002186416.html Accessed Dec. 5, 2010.
37. Han L K, Xu B J, Kimura Y, Zheng Y, Okuda H. Platycodin radix affects lipid metabolism in mice with high fat diet-induced obesity. J Nutr 2000; 130:2760-4.
38. Han L K, Zheng Y N, Xu B J, Okuda H, Kimura Y. Saponins from platycodin radix ameliorate high fat diet-induced obesity in mice. J Nutr 2002; 132:2241-5.
39. Zhao H L, Sims J S, Shim S H, HA Y W, Kang S S, Kim Y s. Antiobese and hypolipidemic effects of platycodin saponins in diet-induced obese rats: evidence for lipase inhibition and calorie restriction. Int J Obes (lond). 2005; 29(8):983-90.
40. Rupnick M A, Panigrahy D, Zhang C Y, Dallabrida S M, Lowell B B, Langer R, Folkman J M. Adipose tissue mass can be regulated through the vasculature. PNAS 2002; 99(16):10703-10735.
41. Lijnen H R, Frederix L, Van Hoef B. Fumagillin reduces adipose tissue formation in murine models of nutritionally induced obesity. Obesity (Silver Spring). 2010 December; 18(12):2241-6.
42. Bråkenhielm E, Cao R, Gao B, Angelin B, Cannon B, Parini P, Cao Y. Angiogenesis inhibitor, TNP-470, prevents diet-induced and genetic obesity in mice. Circ Res. 2004 Jun. 25; 94(12):1579-88.
43. Greenway F L, Liu Z, Yu Y, Caruso M K, Roberts A T, Lyons J, Schwimer J E, Gupta A K, Bellanger D E, Guillot T S, Woltering E A. An assay to measure angiogenesis in human fat tissue. Obes Surg. 2007 April; 17(4):510-5.
44. Woltering E A, Lewis J M, Maxwell P J IV, Frey D J, Wang Y Z, Rothermel J, et al. Development of a novel in vitro human tissue-based angiogenesis assay to evaluate the effect of antiangiogenic drugs. Ann Surg 2003; 237:790-798. [PubMed: 12796575] discussion 798-800
45. Hornick C A, Myers A, Sadowska-Krowicka H, Anthony C T, Woltering E A. Inhibition of angiogenic initiat ion and disruption of newly established human vascular networks by juice from Morinda citrifolia (noni). Angiogenesis 2003; 6:143-149. [PubMed: 14739620]
46. Liu Z, Schwimer J, Liu D, Greenway F L, Anthony C T, Woltering E A. Black raspberry extract and fractions contain angiogenesis inhibitors. J Agric Food Chem. 2005 May 18; 53(10):3909-15.
47. Liu Z, Schwimer J, Liu D, Lewis J, Greenway F L, York D A, Woltering E A. Gallic acid is partially responsible for the antiangiogenic activities of Rubus leaf extract. Phytother Res. 2006 September; 20(9):806-13.
48. York D A, Thomas S, Greenway F L, Liu Z, Rood J C. Effect of an herbal extract Number Ten (NT) on body weight in rats. Chin Med. 2007 Sep. 14; 2:10.
49. Greenway F L, Liu Z, Martin C K, Kai-yuan W, Nofziger J, Rood J C, Yu Y, Amen R J. Safety and efficacy of NT, an herbal supplement, in treating human obesity. Int J Obes (Lond). 2006 December; 30(12):1737-41.
50. Roberts A T, Martin C K, Liu Z, Amen R J, Woltering E A, Rood J C, Caruso M K, Yu Y, Xie H, Greenway F L. The safety and efficacy of a dietary herbal supplement and gallic acid for weight loss. J Med. Food. 2007 March; 10(1):184-8.
51. Glick Z. Modes of action of gallic acid in suppressing food intake of rats. J. Nutr. 1981 November; 111(11):1910-6.
52. Booth A, Amen R J, Scott M, Greenway F L. Oral dose-ranging developmental toxicity study of an herbal supplement (NT) and gallic acid in rats. Adv Ther. 2010 April; 27(4):250-5.

We claim:

1. A method of ameliorating or inhibiting angiogenesis associated with a disease condition in a mammal, said method comprising administering to the mammal an effective amount of an extract of Platycodin radix containing platycodin D as an active ingredient; wherein the disease condition is selected from the group consisting of diabetic retinopathy, macular degeneration, sickle cell anemia, sarcoidosis, syphilis, pseudoxanthoma elasticum, Crohn's disease, Paget's disease, vein occlusion, artery occlusion, carotid obstructive disease, chronic uveitis/vitritis, mycobacterial infections, Lyme disease, systemic lupus erythematosis, psoriasis, Kaposi's sarcoma, rheumatoid arthritis, uveitis, retinopathy of prematurity, Eales disease, Bechets disease, infections causing retinitis or choroiditis, presumed ocular histoplasmosis, Bests disease, myopia, optic pits, Stargarts disease, pars planitis, chronic retinal detachment, hyperviscosity syndrome, toxoplasmosis, trauma, and post-laser surgery complications.

2. The method of claim 1, wherein the extract of Platycodin radix is administered orally.

3. The method of claim 1, wherein the extract of Platycodin radix is administered topically.

4. The method of claim 1, wherein the extract of Platycodin radix is administered parenterally.

5. The method of claim 1, wherein the disease condition is psoriasis and the administration of the extract of Platycodin radix is topical or oral.

6. The method of claim 1, wherein the mammal is a human.

7. The method of claim 1, wherein the extract contains platycodin D from about 20% to about 40% by weight.

8. A method of ameliorating or inhibiting angiogenesis associated with a disease condition in a mammal, said method comprising administering to the mammal an effective amount of platycodin D; wherein the disease condition is selected from the group consisting of diabetic retinopathy, macular degeneration, sickle cell anemia, sarcoidosis, syphilis, pseudoxanthoma elasticum, Crohn's disease, Paget's disease, vein occlusion, artery occlusion, carotid obstructive disease, chronic uveitis/vitritis, mycobacterial infections, Lyme disease, systemic lupus erythematosis, psoriasis, Kaposi's sarcoma, rheumatoid arthritis, uveitis, retinopathy of prematurity, Eales disease, Bechets disease, infections causing retinitis or choroiditis, presumed ocular histoplasmosis, Bests disease, myopia, optic pits, Stargarts disease, pars planitis, chronic retinal detachment, hyperviscosity syndrome, toxoplasmosis, trauma, and post-laser surgery complications.

9. The method of claim 8, wherein the platycodin D is administered orally.

10. The method of claim 8, wherein the platycodin D is administered topically.

11. The method of claim 8, wherein the platycodin D is administered parenterally.

12. The method of claim 8, wherein the disease condition is psoriasis and the administration of platycodin D is topical or oral.

13. The method of claim 8, wherein the mammal is a human.

14. A method of ameliorating or inhibiting angiogenesis associated with a disease condition in a mammal, said method comprising administering to the mammal an effective amount of one or more platycodin saponins; wherein the disease condition is selected from the group consisting of diabetic retinopathy, macular degeneration, sickle cell anemia, sarcoidosis, syphilis, pseudoxanthoma elasticum, Crohn's disease, Paget's disease, vein occlusion, artery occlusion, carotid obstructive disease, chronic uveitis/vitritis, mycobacterial infections, Lyme disease, systemic lupus erythematosis, psoriasis, Kaposi's sarcoma, rheumatoid arthritis, uveitis, retinopathy of prematurity, Eales disease, Bechets disease, infections causing retinitis or choroiditis, presumed ocular histoplasmosis, Bests disease, myopia, optic pits, Stargarts disease, pars planitis, chronic retinal detachment, hyperviscosity syndrome, toxoplasmosis, trauma, and post-laser surgery complications.

15. The method of claim 14, wherein the extract of Platycodin radix is administered orally.

16. The method of claim 14, wherein the extract of Platycodin radix is administered topically.

17. The method of claim 14, wherein the extract of Platycodin radix is administered parenterally.

18. The method of claim 14, wherein the disease condition is psoriasis and the administration of the extract of Platycodin radix is topical or oral.

19. The method of claim 14, wherein the mammal is a human.

20. The method of claim 14, wherein the one or more platycodin saponins are selected from the group consisting of platycodin D, platycodin D3, polygalactin D, platycodin A, acetyl-polygalactin D, prosapogenin D, prosapogenin D methyl ester, 2″-O-acetyl polygalactin D, Platycodin A, prosapogenin, and prosapogenin D methyl ester.
